(19) United States
(12) Patent Application Publication
(10) Pub. No.: US 2026/0165644 A1
(43) Pub. Date: Jun. 18, 2026

(54) METHOD AND SYSTEM FOR SELECTIVE TETANIC STIMULUS IN PATIENT MONITORING

(71) Applicant: GE Precision Healthcare LLC, Waukesha, WI (US)

(72) Inventor: Mika Olli Kristian Särkelä, Uusimaa (FI)

(21) Appl. No.: 18/984,780

(22) Filed: Dec. 17, 2024

(51) Int. Cl. A61B 5/00; A61B 5/395

(52) U.S. Cl. CPC A61B 5/4821; A61B 5/395

(57) ABSTRACT

Methods and systems are provided for monitoring neuromuscular blockade and analgesic administration in patients before, during, and after surgical procedures. In one embodiment, a method includes applying a tetanic stimulus to a nerve of a patient for a preset duration via a peripheral nerve stimulator of a patient care and monitoring system, measuring and registering an initial physiological response of the patient at a start of the tetanic stimulus via a physiological response monitor of the patient care and monitoring system, monitoring a physiological response of the patient periodically within the preset duration via the physiological response monitor, and automatically preemptively aborting the tetanic stimulus administered by the peripheral nerve stimulator based on a comparison of the physiological response to the initial physiological response.

### FIELD

Embodiments of the subject matter disclosed herein relate to medical devices, and more particularly, to monitoring physiological response to nerve stimulation before, during, and after a surgical procedure.

### BACKGROUND

Neuromuscular transmission (NMT) is the transfer of an impulse between a nerve and a muscle in a neuromuscular junction. Patients are typically treated with neuromuscular blocking agents (NMBA) prior to and/or during surgery to block NMT and paralyze skeletal muscles by preventing nerve impulses from passing through a respective neuromuscular junction. Muscle relaxation induced by NMBA may be used to enable endotracheal intubation and provide desirable working conditions for a surgical procedure. A level of neuromuscular block may be monitored to ensure appropriate block is provided before and during a procedure and/or to determine when the patient can be extubated (i.e., removal of endotracheal tube). For example, post-surgical NMBA clearance is monitored to determine when neuromuscular receptors of the patient are no longer blocked by the NMBA and thus respiratory support provided to the patient may be removed.

Modern general anesthesia also includes analgesic drugs, such as opioids, that cause antinociception (e.g., block the pain pathways of the patient). Administration of analgesic drugs at an adequate dosage may minimize nociceptive responses of the patient to surgical stimuli, where nociceptive responses may be painful and/or stressful to the patient. Minimizing and avoiding nociceptive responses of the patient may accelerate post-operative recovery. However, predicting an adequate analgesic drug dose prior to surgery may be difficult as there is no tool for the purpose. Therefore, dose of analgesic medication may be adjusted several times during the surgery in response to patient nociceptive responses, such as hemodynamic change, tearing, sweating, and so on.

### BRIEF DESCRIPTION

In one embodiment, a method includes applying a tetanic stimulus to a nerve of a patient for a preset duration via a peripheral nerve stimulator of a patient care and monitoring system, measuring and registering an initial physiological response of the patient at a start of the tetanic stimulus via a physiological response monitor of the patient care and monitoring system, monitoring a physiological response of the patient periodically within the preset duration via the physiological response monitor, and automatically preemptively aborting the tetanic stimulus administered by the peripheral nerve stimulator based on a comparison of the physiological response to the initial physiological response.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a block diagram of an exemplary patient care and monitoring system;

FIG. 2 shows further detail of the exemplary patient care and monitoring system of FIG. 1;

FIG. 3 is a flow chart of a method for selective administration of tetanic stimulus to monitor NMT;

FIG. 4 shows a graph illustrating NMT measurement modalities at different ACh receptor block levels;

FIG. 5 is a flow chart of a method for selective administration of tetanic stimulus to monitor patient recovery from NMBA;

FIG. 6 is a temporal graph illustrating muscle activity in response to tetanic stimulus;

FIG. 7 is a flow chart for selective administration of tetanic stimulus to monitor patient sedation by administration of analgesic medication;

FIG. 8 is a first graph of vasoconstriction reaction in response to tetanic stimulus;

FIG. 9 is a second graph of vasoconstriction reaction in response to tetanic stimulus; and

FIG. 10 is a temporal graph illustrating nociceptive response to tetanic stimulus.

### DETAILED DESCRIPTION

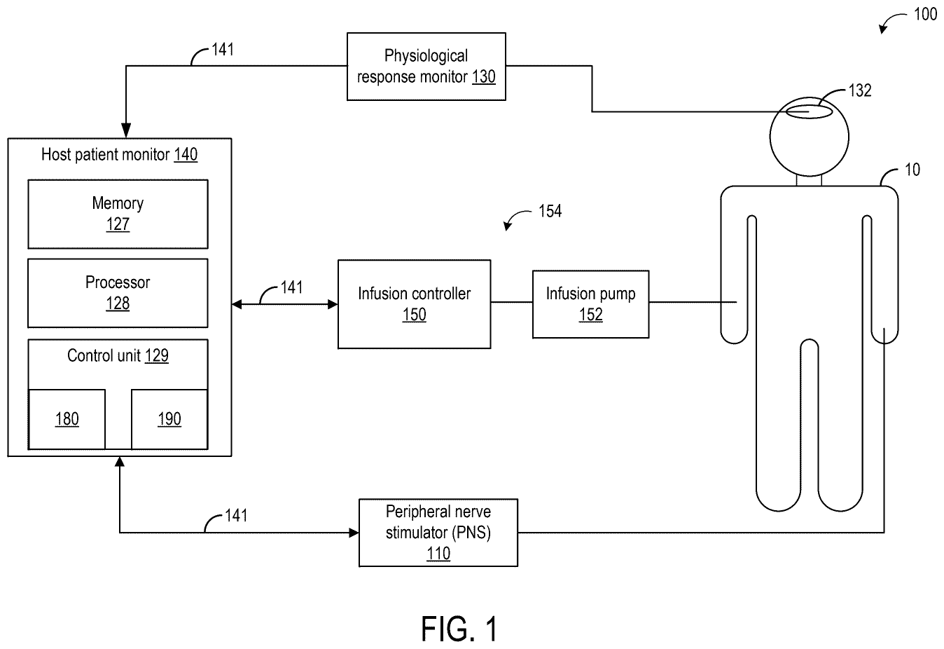

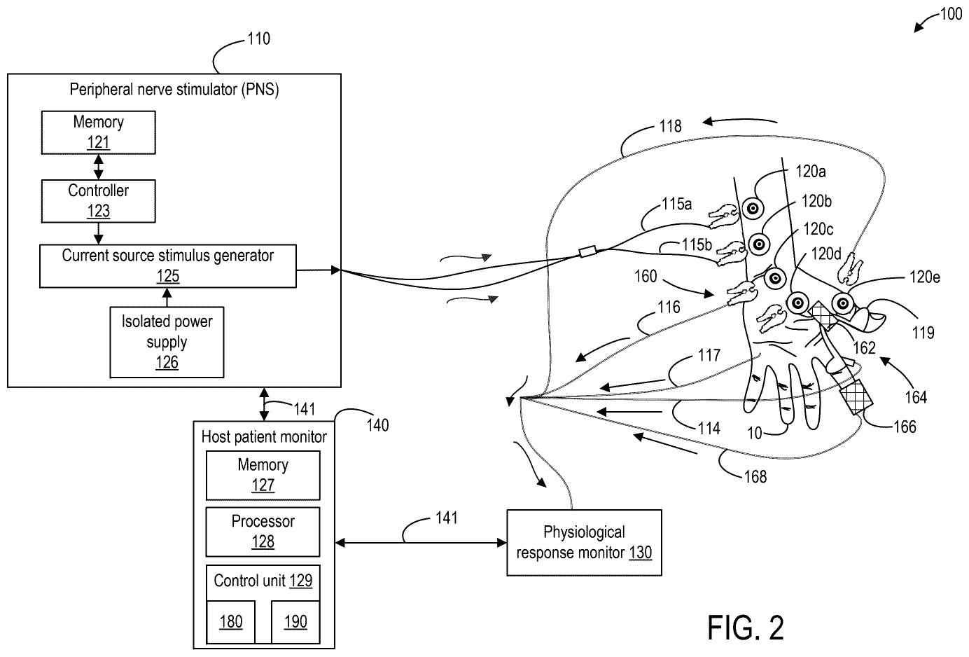

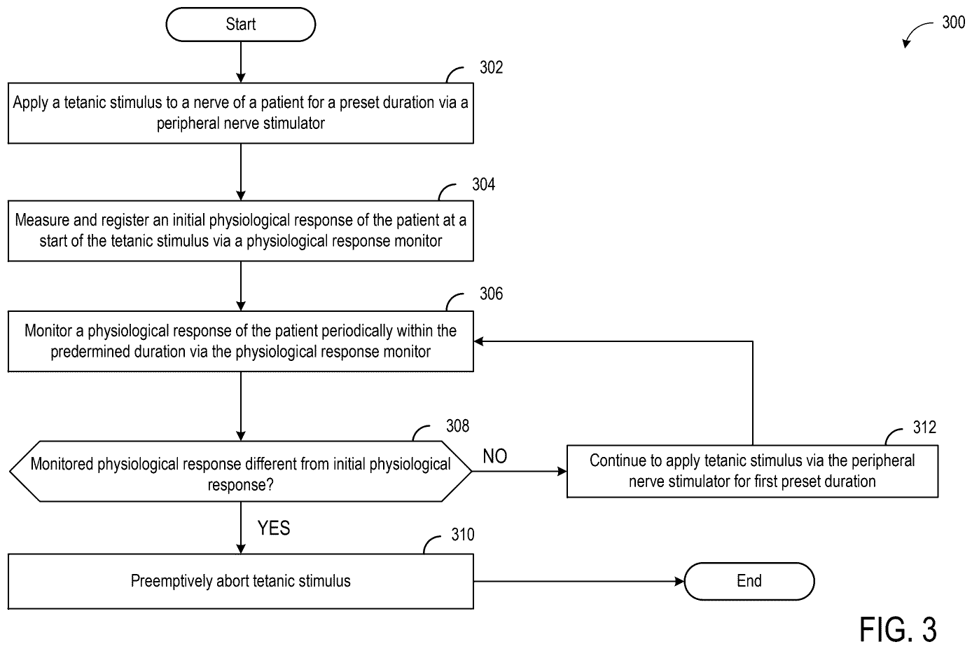

The following description relates to various embodiments of systems and methods for selective application of tetanic stimulus in patient monitoring, as may be used before, during, and after surgical procedures to monitor an amount of neuromuscular blockage after the administration of muscle relaxants in patients during surgery. FIGS. 1 and 2 show an exemplary patient care and monitoring system that may be used for selective application of tetanic stimulus and real-time monitoring of patient response to tetanic stimulus. The patient care and monitoring system may provide multiple applications for monitoring physiological response to nerve stimulation. For example, the patient care and monitoring system may be used as a NMT measurement modality to monitor residual relaxation of a patient. The patient care and monitoring system may further be used as a nociceptive response monitoring modality. FIG. 3 is a flow chart of a method that may be implemented by the patient care and monitoring system for selective tetanic stimulus in patient monitoring and specifically, monitoring physiological response to nerve stimulation. The method of FIG. 3 may be used to monitor NMT and/or nociceptive response.

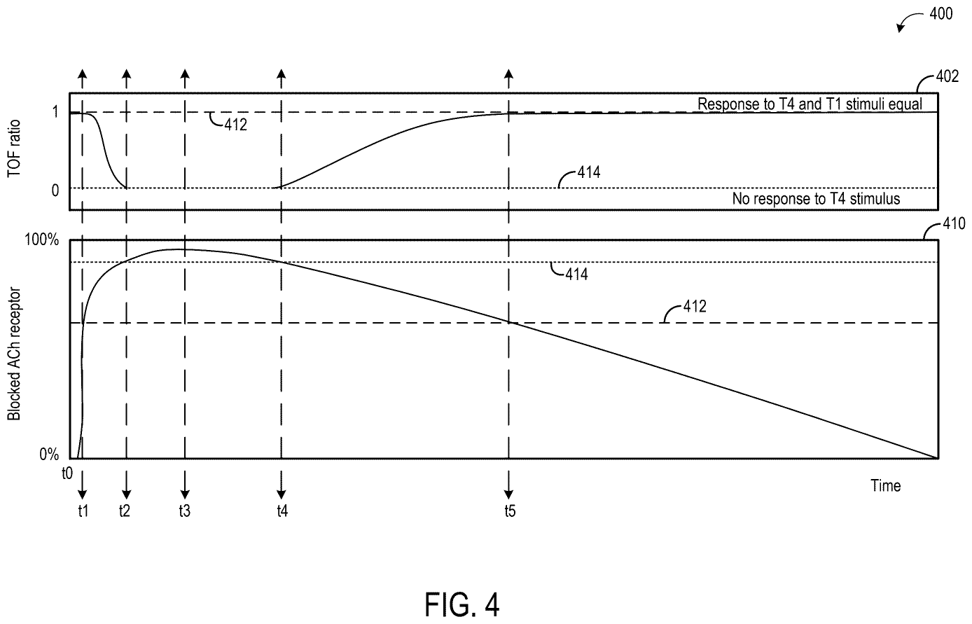

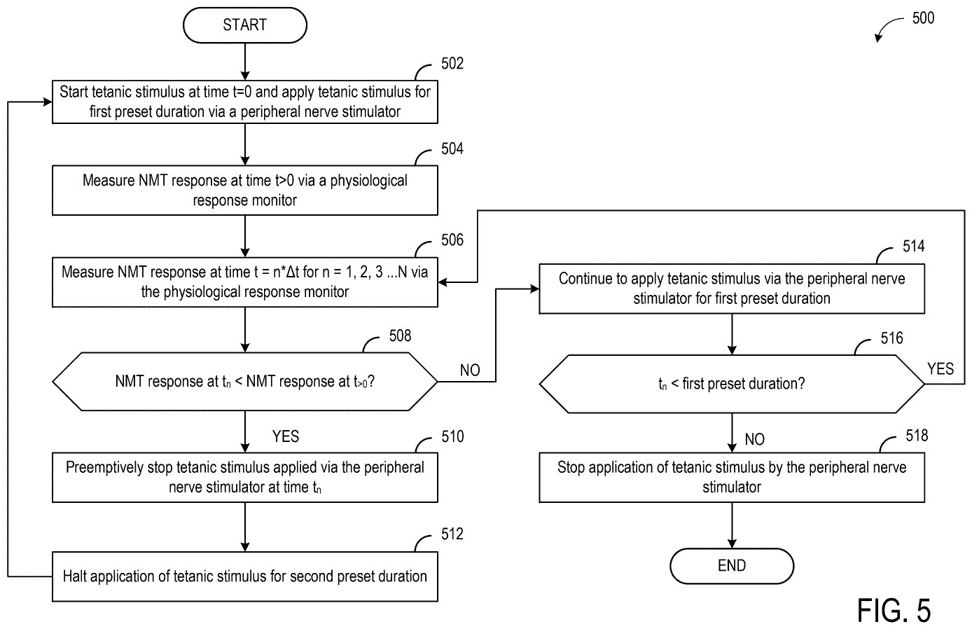

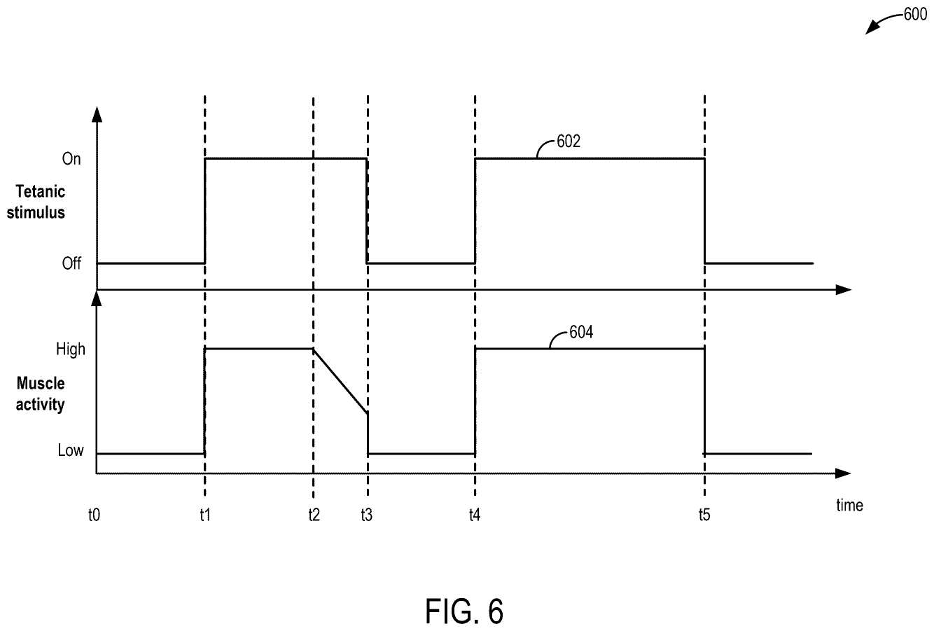

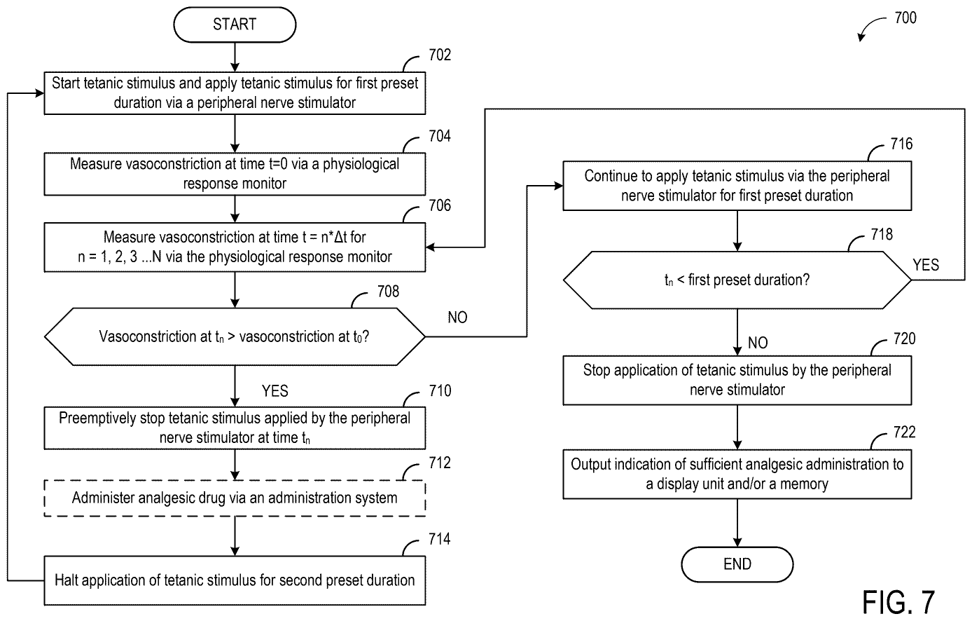

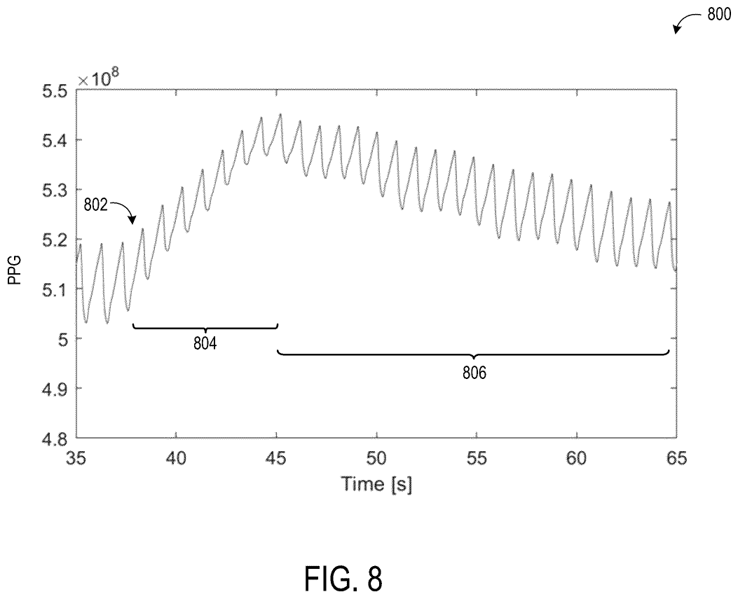

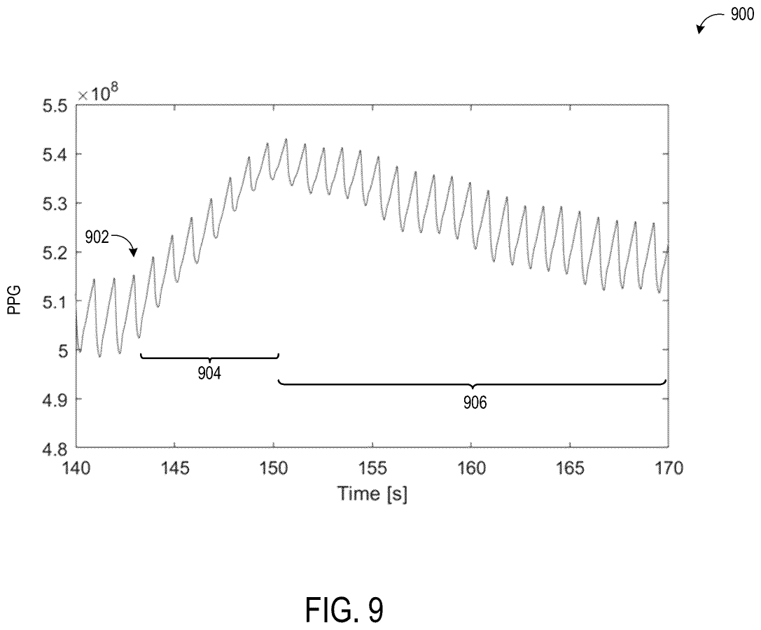

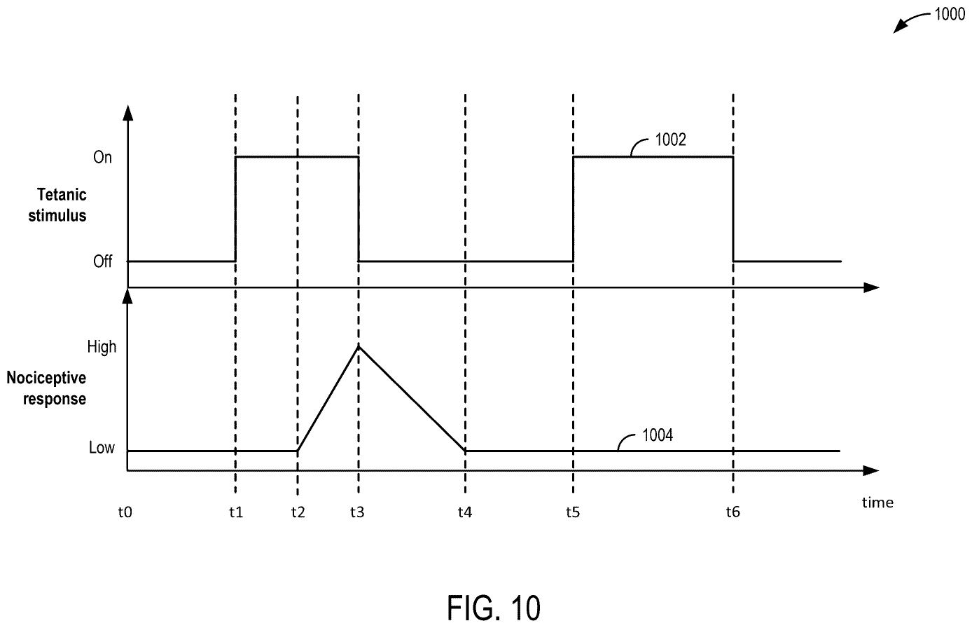

Efficacy of conventional methods for monitoring NMT at different ACh receptor block levels are shown in graphs of FIG. 4. A method illustrated in a flow chart of FIG. 5 may be implemented by the patient care and monitoring system of FIGS. 1-2 to monitor patient recovery from NMBA (e.g., to monitor NMT). FIG. 6 is a temporal graph illustrating muscle activity in response to tetanic stimulus, and may be an example of execution of the method of FIG. 5. FIG. 7 is a flow chart illustrating a method for monitoring patient analgesia by administration of analgesic medication that may be implemented by the patient care and monitoring system of FIGS. 1-2 (e.g., to monitor nociceptive response). FIGS. 8 and 9 show graphs of vasoconstriction reaction in response to tetanic stimulus selectively administered as described with respect to FIG. 7. FIG. 10 is a temporal graph illustrating nociceptive response to tetanic stimulus, and may be an example of execution of the method of FIG. 7.

Conventional practices for monitoring neuromuscular transmitter (NMT) activity and recovery following administration of NMBA to a patient includes using train-of-four (TOF) to monitor recovery of muscle activity. TOF monitoring includes stimulating a muscle group with four consecutive brief (0.2-0.3 millisecond (ms)) stimuli, and measuring a number of resulting muscle activations (e.g., twitches) to determine if the patient is recovered/recovering from neuromuscular blockage. However, TOF is a non-optimal tool for monitoring residual muscle relaxation. According conventional guidelines, the patient may be extubated once NMT TOF ratio has exceeded 90%. However, there is no strong evidence that this would reduce incidence of post-operative respiratory complications of the patient. One possible explanation may be that at the level of TOF 90%, there is still significant residual neuromuscular block, as around 70% of the acetylcholine (ACh) receptors are still blocked. Thus, a maximum ability of TOF monitoring is reached when approximately 70% of neuromuscular receptors may still be blocked. It may be desirable for the patient to remain intubated (i.e., having endotracheal tube inserted) and/or to continue NMT monitoring after extubation. However, the dynamic range TOF measurement does not enable NMT monitoring in the ACh block range of 70% and below.

In the ACh block range of 70% and below, tetanic fade monitoring may be used as a NMT measurement modality and for monitoring residual muscle relaxation. During tetanic fade monitoring, a tetanic stimulus is applied to the patient, where the tetanic stimulus may stimulate muscle activity. The tetanic stimulus may be applied for a preset duration, such as five seconds. It may be desirable for tetanic stimulus to be as short (e.g., five seconds or less) as possible, as it is known that longer tetanic stimuli are more stressful to the patient than shorter ones. The tetanic stimulus may stimulate an initial level of muscle activity at a time after the tetanic stimulus is first applied. For example, if muscle activity is measured by electrical activity of muscle using electromyography (EMG), there may be a delay (e.g., approximately 3 milliseconds (ms) from nerve stimulation (e.g., at time (t)=0 ms) to an onset of muscle activation. After the onset of muscle activation, it may take an additional duration for a maximum muscle activation level is reached. Muscle activity may additionally or alternatively be monitored using one or more of mechanomyography (MMG), phonomyography (PMG), kinemyography (KMG), acceleromyography (AMG). In MMG, PMG, KMG, and AMG monitoring, a delay between nerve stimulation and muscle activation may be longer than for EMG monitoring (e.g., longer than 3 ms). For example, the initial level of muscle activity may be obtained between 3 ms and 20 ms.

Muscle activity is monitored during application of the tetanic stimulus (e.g., during the preset duration). A decrease in muscle activity (e.g., also referred to as “tetanic fade”) during the preset duration may indicate that muscles of the patient are still relaxed (e.g., corresponding neuromuscular receptors are still blocked by NMBA). It may be desirable to provide and/or continue to provide the patient with respiratory support (e.g., mechanical ventilation via endotracheal tube or laryngeal mask; or continuous positive airway pressure (CPAP) via face mask). A desirable physiological response for monitoring NMBA recovery may be that there is no decrease in muscle activity for the preset duration (e.g., no tetanic fade). When the patient is recovering from NMT receptor blockage (e.g., after a surgical procedure), physiological responses such as continuous muscle activity may indicate that NMT receptors are sufficiently free. If the muscle activity does not decrease during the preset duration (e.g., muscle activity is equal to the initial level of muscle activity during the preset duration), it is determined that the neuromuscular receptors of the patient are no longer blocked. The patient may be removed from respiratory support.

Tetanic stimulus may also be used to determine if an adequate dose of analgesic medication is administered to a patient prior to a surgical procedure. For example, the amount of analgesic medication may be adjusted based on the patient hemodynamic response to tetanic stimulus given before the surgery. When the patient is being administered analgesic drugs (e.g., before and/or during a surgical procedure), physiological responses such as an increase in nociceptive response during tetanic stimulus may indicate that a present analgesia dose may not sufficiently block a nociceptive response of the patient. A desirable physiological response for monitoring analgesia administration is a lack of nociceptive response. If there is a response to tetanic stimulus, analgesia may be increased. If there is no response analgesia is adequate and surgery can start.

While tetanic fade provides sufficient monitoring of residual muscle relaxation following use of TOF monitoring, tetanic stimulus may be painful and/or uncomfortable to the patient, especially when the patient is awake or waking up during application of the tetanic stimulus. Tetanic stimulus may cause hemodynamic response in the patient, such as increased vasoconstriction, which is a sign of stressful and/or painful event experienced by the patient. Tetanic stimulus is a nociceptive event that triggers catecholamine (such as epinephrine, norepinephrine) release within a body and consequently may increase blood pressure and heart rate of the patient. Therefore, the use of tetanic stimulus on awake or moderately sedated patients is typically avoided. A method for monitoring residual muscle relaxation is desired that avoids stressful and/or painful events for the patient.

Described herein are systems and methods for patient monitoring that selectively use tetanic stimulus before, during, and after administration of neuromuscular blocking agents and/or analgesic drugs to monitor physiological response of the patient. A tetanic stimulus is applied for a preset duration (e.g., 5 seconds). Physiological responses of the patient are monitored in real-time with the on-going tetanic stimulus. Physiological responses can be based on one or more of EMG signal, MMG signal, PMG signal, KMG signal, AMG signal, photoplethysmographic (PPG) pulse wave signal (AC & DC), or other physiogical signal with fast response time. If an undesired physiological response (e.g., tetanic fade, muscle activity, vasoconstriction in PPG signal) is detected within the preset duration of tetanic stimulus application, tetanic stimulus is automatically aborted preemptively (e.g., before the end of the preset duration) before further stress is caused to the patient. Compared to conventional uses of tetanic stimulus to monitor physiological responses of a patient, the system and method described herein may decrease stress and/or pain experienced by the patient because the tetanic stimulus is automatically preemptively aborted in response to detection of a physiological response, rather than the tetanic stimulus being administered for an entire predetermined stimulus duration. Further, automatically stopping tetanic stimulus application in real-time (e.g., when the physiological response is detected) may reduce stress and/or pain experienced by the patient compared to manual stoppage of the tetanic stimulus (e.g., by a user), which may be slower than automatic stoppage.

For applications of the systems and methods to monitor NMBA recovery, automatic preemptive abortion of the tetanic stimulus may be followed by a preset rest period in which no tetanic stimulus is applied. During the rest period, the patient may further recover from NMBA blockage (e.g., NMBA may vacate NMT receptors). For example, possible vasoconstriction caused by tetanic stimulus can be monitored with a PPG device. If vasoconstriction is observed during the tetanic stimulus, tetanic stimulus is automatically preemptively aborted, and a non-opioid analgesic drug (e.g., dexmedetomidine) may be administered for facilitating patient recovery. Following the rest period, tetanic stimulus may again be applied for the preset duration, and automatically aborted preemptively in response to detection of an undesired physiological response. The method may be repeated until no undesirable physiological response is detected during the preset duration.

For applications of the systems and methods to monitor NMBA and analgesia administration, automatic preemptive abortion of the tetanic stimulus may be followed by a preset rest period, as described above. During the preset rest period, the additional analgesia and/or NMBA may be administered to the patient.

When the systems and methods described herein are used to monitor recovery from NMBA administration, patient pain and stress may be decreased, and post-operative recovery may be increased. In conventional uses of tetanic stimulus to monitor recovery from NMBA administration, tetanic stimulus may be manually stopped in response to detection of a physiological response by the patient to the stimulus (e.g., visually by a user). In some examples, the tetanic stimulus may not be stopped and may be administered for a full preset duration. The NMT receptors of the patient may not be fully blocked (e.g., the patient may be at least partially recovered from NMBA administration), however the patient may not exhibit physiological responses that are discernable by a user. Automatic, physiological response-triggered preemptive abortion of tetanic stimulus enables assessment of residual neuromuscular block that causes less stress and/or pain for a patient compared to continuous (e.g., five seconds long) tetanic stimulus. Residual neuromuscular block may be monitored while causing the patient to experience less stress hormone release (e.g., epinephrine, norepinephrine), which may facilitate faster and more successful patient recovery compared to conventional monitoring methods using tetanic stimulus. This may make tetanic fade more feasible method for routine clinical use, including use on nearly-awake patient.

When the systems and methods described herein are used to monitor analgesia administration, accurate and appropriate administration of analgesic drugs may be obtained. This may further reduce patient pain and/or stress during post-operative recovery as a result of reduced stress during the procedure. Analgesic drug administration is a trade-off between the drug side effects and a nociceptive/stressful response attenuated by the drug. When the adequacy of analgesic level is checked before the surgery with the help of tetanic stimulus, it is likely that there will be less nociceptive responses during the surgery and less demand to adjust analgesia afterwards.

Attempts to apply the disclosed methods manually by a user, where tetanic stimulus is manually stopped in response to manual identification of nociceptive response, is insufficient. A reaction time of a user may not be fast enough to realize the patient is responding to tetanic stimulus and then stop the tetanic stimulus, which may cause the patient stress and/or pain and may increase and/or complicate patient recovery. Further, the methods described herein may be more sensitive to physiological (e.g., nociceptive, tetanic fade) responses than a user. Even if a method indicates that an undesired physiological response is occurring, and a user then stops the tetanic stimulus, the reaction time may be long enough to cause pain and/or stress to the patient. The method for automatic shutoff decreases this reaction time and removes potential for user error, for example, not knowing how to interpret the data and therefore not knowing when to stop tetanic stimulus, improperly stopping tetanic stimulus (e.g., not all the way off), and/or restarting tetanic stimulus too early after a rest period.

FIG. 1 shows a block diagram of an exemplary patient care and monitoring system 100 configured to selectively apply a tetanic stimulus to a patient and, in real-time, monitor a physiological response of the patient to the tetanic stimulus. The patient care and monitoring system 100 includes a peripheral nerve stimulator (PNS) 110, a physiological response monitor 130, and an administration system 154, each of which are communicably coupled to a host patient monitor 140 via a communication link 141. The communication link 141 may be a wired and/or a wireless connection. In a wireless embodiment, the PNS 110 and/or the physiological response monitor 130 may have a wireless receiver/transmitter configured to wirelessly communicate with the host patient monitor 140. In such an embodiment, the communication link 141 being a wireless communication link, may be via any of various wireless protocols, such as Bluetooth, Bluetooth low energy (BLE), ZigBee, or may be via the wireless medical telemetry service (WMTS), to provide just a few examples.

The PNS 110, the physiological response monitor 130, and the administration system 154 may further be coupled to a patient 10 to administer tetanic stimulus, monitor response to tetanic stimulus, and supply analgesic drugs to the patient 10, respectively. The PNS 110 is configured to control and transmit tetanic stimulation, as further described with respect to FIG. 2.

The physiological response monitor 130 is configured to record a physiological response of the patient 10 via one or more monitoring probes, electrodes, or other monitoring device 132. The physiological response monitor 130 may further be configured to monitor one or more types of physiological response via one or more monitoring methods. For example, the physiological response monitor 130 may be configured to monitor NMT based on neuromuscular activity and depth of muscle relaxation, such as via EMG and/or KMG techniques. The physiological response monitor 130 may additionally or alternatively be configured for one or more of MMG, PMG, AMG, and/or PPG pulse wave monitoring. Further, the physiological response monitor 130 may be configured to monitor vasoconstriction and thus nociceptive response of a patient. For example, the physiological response monitor 130 may be configured as and/or include a SpO₂ device, a PPG pulse wave monitor, and/or another type of nociceptive response monitoring device. The patient care and monitoring system 100 may include more than one physiological response monitor 130 coupled to the host patient monitor 140 and the patient 10, where each physiological response monitor 130 is configured to monitor a different physiological response. A type of physiological response monitor 130 may be configurable (e.g., selectively coupled to the host patient monitor 140 and/or the patient 10) in response to demands of a given patient and/or procedure. For example, the physiological response monitor 130 may be an EMG monitor that is configured to record EMG potentials from the patient 10 via one or more EMG electrodes (e.g., monitoring device 132), such as an electrode strip configured to attach to the patient's forehead, and to communicate with the host patient monitor via the communication link 141.

The administration system 154 may include an infusion controller 150 configured to control one or more infusion pumps 152 that are in turn configured to administer NMBA and/or analgesic drugs to the patient 10. The infusion controller 150 may be configured to determine and/or administer desired dose of a given drug and/or NMBA. For example, as further described with respect to FIGS. 7-10, the infusion controller 150 may be configured to automatically determine when initiation of analgesic drug administration is desired, and to automatically begin analgesic drug administration in response to detection of a nociceptive response by the physiological response monitor 130.

Host patient monitor 140 may include a memory 127, a processor 128, and a control unit 129. Memory 127 may have similar functions as memory 121. Control unit 129 may include control buttons/knobs 180 and a display unit 190. The control buttons and knobs of control unit 129 may be configured to allow for user input. The display unit 190 may be configured to receive touch input from a user.

FIG. 2 illustrates further detail of the patient care and monitoring system 100, including elements of the PNS 110 for selectively administering tetanic stimulus, and elements of the physiological response monitor 130 used to monitor response to tetanic stimulus. In some examples, the physiological response monitor 130 may be at least partially integrated in the PNS 110.

The PNS 110 is coupled to a plurality of neurostimulators, 115a and 115b, for providing stimulation output (e.g., electrical stimuli) of varying type and frequency to the patient 10. In the depicted example, neurostimulators 115a and 115b are connected to stimulating electrodes 120a and 120b, respectively, which may apply an electrical stimulus to the patient's ulnar nerve at a pre-determined time interval. The amount of electrical stimulation provided to the neurostimulators is controlled by a current stimulus generator which receives command signals from a controller 123. The controller 123 is linked to a current stimulus generator 125 and selectively controls the current stimulus generator 125 to use power from an isolated power supply 126 to administer a tetanic stimulus. The type and frequency of the stimulation output may be adjusted manually by the user (manual mode) or be automatically chosen by the system (automatic mode). In one example, the type and frequency of the stimulation output may be adjusted by the user via pressing buttons or knobs 180 on the host patient monitor 140. In one example, neurostimulators 115a and 115b may be two wires of positive and negative charges, which may be attached by alligator clips to stimulating electrodes 120a and 120b on the skin of the patient's forearm.

A power supply (not shown) may supply electricity to an isolated power supply 126 which in turn provides power to current stimulus generator 125. The controller 123 may be connected to the current stimulus generator 125 to adjust the amount of electric current provided to the neurostimulators 115a-b. The current stimulus generator 125 may selectively generate tetanic stimulus. Further, the types of neurostimulation may be chosen via a manual or an automatic stimulating mode. If a manual stimulating mode is chosen, then the user may input the desired neuromuscular stimulating types, current range, and pulse width and/or frequency via pressing button 180 of the host patient monitor 140, for example. Alternatively, if a touch-screen is used as the display unit (e.g., display unit 190 of host patient monitor 140), then user input may be provided via touch input to the touch-screen on the display unit.

The physiological response monitor 130 includes one or more transducers for monitoring the evoked physiological response to the electrical stimuli provided by the neurostimulators 115a-b of the PNS 110. For example, the physical response monitor 130 may be configured as and/or include an EMG sensor 160 comprising a plurality of electro-sensors for measuring the action potential of muscle contraction in response to nerve stimulation. The physiological response monitor 130 may additionally or alternatively include and/or be configured as a KMG sensor 164 comprising of mechano-sensor for measuring limb movement in response to nerve stimulation of muscle activity. The signals detected by the transducers may then be converted into electrical signals by an A/D converter (not shown) of PNS 110. The EMG sensor 160 and the KMG sensor 164 are described herein as example NMT monitoring elements of the physiological response monitor 130. The physiological response monitor 130 may additionally or alternatively include a MMG sensor, a PMG sensor, and/or an AMG sensor.

EMG sensor 160 may include a plurality of electro-sensing connections 116, 117, and 118 connected to sensing electrodes 120c, 120d, and 120e, respectively. Most commonly, the three sensing electrodes are positioned to give the most uninterrupted EMG signals. In the depicted example, sensing electrode 120e is placed over the muscle tendon or finger, sensing electrode 120d is placed over the mid-portion of the muscle close to thumb, while sensing electrode 120c may be variable. In one example, electrodes 120d and 120e may be recording electrodes, while electrode 120c may be a grounding electrode. The grounding electrode provides a common reference for the EMG recording electrodes. EMG sensor 160 measures the magnitude of electrical activity sensed by electrodes 120c-120e in response to nerve stimulation and when received at the physiological response monitor 130, is recorded as the EMG muscle response signal.

KMG sensor 164 includes a mechano-sensing connection 114 connected to bending element 119. In the depicted example, bending element 119 is placed between the thumb and the forefinger held in place by an elastic tape 162. The bending element 119 may comprise a piezoelectric polymer film which creates an electrical current in response to movement of any part of the polymer. When compressed or distorted, piezoelectric materials produce a charge proportional to the degree of alteration in shape. In FIG. 1, when the motor nerve is stimulated, thumb movement may cause a shape distortion in bending element 119 which in turn produces an electrical signal transmitted by mechano-sensing connection 114 and recorded at the physiological response monitor 130 as a KMG muscle response signal.

In further examples, the physiological response monitor 130 may be configured as and/or include one or more devices configured to monitor nociceptive response, in addition to and/or instead of one or more NMT monitors (e.g., the EMG and/or the KMG sensors 160, 164). For example, the physiological response monitor 130 may include a pulse oximeter 166, also referred to herein as a SpO₂ monitor, that may be coupled to the patient 10 at the forefinger and coupled to the physiological response monitor 130 via a connection 168. The physiological response monitor 130 may further include a nociceptive monitor that is not in contact with the patient 10, such as a thermal sensor.

Information regarding the NMT and nociceptive response detected using the physiological response monitor 130 may be sent to the host patient monitor 140 via a communication link 141, which may be a wired and/or wireless connection. The processor 128 of the host patient monitor 140, may execute instructions for a method stored in the memory 127 that compares a physiological response received via the physiological response monitor 130 to an initial physiological response detected by the physiological response monitor 130 at a start of tetanic stimulus administration by the PNS 110. In response to the received physiological response differing from the initial physiological response, the processor 128 transmits a signal to the PNS 110 to automatically preemptively aborting the tetanic stimulus. For example, the host patient monitor 140 may transmit a signal to the controller 123 of the PNS 110 to halt generation of the tetanic stimulus by the current stimulus generator 125.

In one example, muscle response signals from EMG sensor 160 and KMG sensor 164 may be differentiated and further fed into a signal scaling and filtering circuit (not shown). After scaling the signal and filtering noise, the signal may be converted from an analog signal to a digital signal in analog-to-digital (A/D) converter and sent to processor 128 of the host patient monitor 140 for processing. Further, the muscle response signals may also be amplified via an amplifier (not shown) before being transmitted into the A/D converter. In one example, the processed signals may be transmitted to the host patient monitor 140 and displayed on the display unit 190 in real-time. Further still, the processed signals may be updated and stored in memory 121. Memory 121 may be a conventional microcomputer which includes: a central processing unit (CPU), a read-only memory (ROM), a random access memory (RAM), and a conventional data bus. Memory 121 may further include a look-up table to convert neuromuscular response value between different stimulus modes.

Control unit 129 may also include a user interface (not shown) which can be used to control operation of the patient care and monitoring system 100, including controlling the input of patient data, changing the monitoring parameters (e.g. stimulus type, current range, frequency/pulse width, etc.), and the like. The user interface may also include a graphical user interface configured for display on a display device, such as display unit 190. The graphical user interface may include information to be output to a user (such as muscle response signals, patient data, etc.) and may also include menus or other elements through which a user may enter input the control unit 129. Further, the processor 128 may process the input provided by the user and command a current stimulus generator 125 to provide a stimulus waveform and current depending on the selected stimulus mode, current range and pulse width/frequency.

FIG. 3 shows a flow chart of a method 300 for real-time physiological monitoring during application of tetanic stimulus, and automatic preemptive abortion of tetanic stimulus in response to changes in physiological response. The method 300 may be carried out according to instructions stored on a computing system, including but not limited to the processor 128 of the patient care and monitoring system of FIGS. 1-2. Method 300 may be executed when sensors of the patient care and monitoring system 100 are securely connected to their respective electrodes on the patient's forearm, such as shown in FIG. 2.

At 302, the method 300 includes applying a tetanic stimulus to a nerve of a patient for a preset duration via a peripheral nerve stimulator (e.g., the PNS 110 of FIGS. 1-2). The tetanic stimulus may have a preset frequency between 50 Hz and 200 Hz, where the same frequency is used for the preset duration. The preset duration may be five seconds. In other examples, the preset duration may be more than or less than five seconds. Tetanic stimulus comprises a series of single stimuli, thus a preset duration of five seconds includes a single five-second-long tetanic stimulus.

At 304, the method 300 includes measuring and registering an initial physiological response of the patient at a start of the tetanic stimulus via a physiological response monitor. The physiological response monitor may be configured to monitor one or more physiological response. For example, the physiological response monitor may be configured to monitor vasoconstriction and thus nociceptive response of a patient (e.g., a SpO2 device, a PPG pulse wave monitor). The physiological response monitor may additionally or alternatively be configured as a NMT monitor including one or more of an EMG, MMG, PMG, KMG, and AMG monitor. The patient care and monitoring system may be configured to monitor in real-time one or more physiological responses while administering tetanic stimulus. For example, the peripheral nerve stimulator may administer tetanic stimulus, and the physiological response monitor (e.g., 130 of FIGS. 1-2) may monitor one or more physiological responses in real-time as tetanic stimulus is administered.

At 306, the method 300 includes monitoring a physiological response of the patient periodically within the preset duration using the physiological response monitor. The physiological response may be monitored at a preset frequency that is a fraction of the preset duration. For example, the preset duration may be 5 seconds. The monitored physiological response may be compared to the initial physiological response in real-time.

At 308, the method 300 includes determining if a monitored physiological response is different from the initial physiological response within the preset duration. Comparison of the initial physiological response and the physiological response during the preset duration in which tetanic stimulus is applied automated further enables automated control decisions in response to the comparison. The automated control decisions enable precise timing of stimulus abortion.

At 308, in response to the monitored physiological response not being different from the initial physiological response (NO at 308), the method 300 proceeds to 312. At 312, the method 300 includes continuing to apply the tetanic stimulus for the preset duration, via the peripheral nerve stimulator. The method 300 returns to operation 306 to continue to periodically monitor the physiological response of the patient.

In response to the monitored physiological response being different from the initial physiological response (YES at 308), the method 300 proceeds to 310. At 310, the method 300 includes automatically preemptively aborting tetanic stimulus, based on the comparison between the monitored physiological response and the initial physiological response. The method 300 ends.

The method 300 may be applied to different NMT monitoring scenarios and is configured to integrate multiple measurement modalities, thus enabling a NMT monitoring system (e.g., the patient care and monitoring system 100 of FIGS. 1-2) to have multiple applications before, during, and after administration of NMBA. For example, the method 300 may be used to monitor patient recovery following administration of NMBA. As described with respect to FIGS. 4-6, the patient care and monitoring system may be used to apply tetanic stimulus and measure a muscle activation in real-time in response to the tetanic stimulus. In this way, the patient care and monitoring system enables NMT monitoring before, during, and after surgical procedures in which NMBA is administered to a patient.

FIG. 4 shows NMT measurement modality graphs 400 that illustrate efficiencies of different NMT measurement modalities at different ACh receptor block levels. A first graph 402 shows a TOF ratio over time, where TOF ratio is plotted on a vertical axis from zero to one and time is plotted on the horizontal axis increasing from left to right. Stimulus is applied at a set frequency (e.g., 100 Hz) starting at t 0 and continuing for the duration shown in the graphs 400. The stimulus applied at each time point may be the same frequency and applied for the same duration. For example, in TOF measurement, four consecutive brief stimuli at 0.5 second intervals are supplied to the nerve, and the evoked activity of each stimulus is measured separately. The TOF ratio is the ratio of the response evoked by a fourth stimulus (T4) to the response evoked by the first stimulus (T1). The TOF ratio zero means there is no response to the fourth stimulus (e.g., no muscle activation detected after the fourth stimulus). The TOF ratio one is a 100% response to TOF stimulus (e.g., the muscle activation in response to the fourth stimulus is equal to the muscle activation in response to the first stimulus). A percentage of TOF response (e.g., TOF ratio) may be used to measure NMT.

A second graph 410 shows a percentage of blocked ACh receptors (plotted on the vertical axis) over time (increasing from left to right along the horizontal axis), where zero is 0% of receptors blocked, and 100% is all receptors blocked. A monitoring margin line 412 is shown on both the first graph 402 and the second graph 410, and illustrates a percentage of ACh receptors blocked at the saturation level of TOF. When the TOF ratio is equal to the monitoring margin line 412 (e.g., at t5) it may be desirable to continue assisted ventilation or otherwise closely monitor the patient, as the patient may be unable to breathe on their own. For example, the monitoring margin line 412 may be approximately 67% of blocked ACh receptors. The monitoring margin line 412 also corresponds with 100% response to TOF stimulation (e.g., response to T4 stimulus equal to response to T1 stimulus). A stimulation response line 414 is also shown on both the first graph 402 and the second graph 410, and illustrates a lower end of TOF ratio dynamic range. There may be no response to the stimulus of TOF stimulation train when a percentage of blocked ACh receptors is approaching 100%. The stimulation response line 414 may be approximately 90% of ACh receptors blocked. The stimulation response line 414 further corresponds with 0% TOF value. The patient may have a response to the first stimulus, the second stimulus, and the third stimulus of the TOF stimulus, but may not have a response to the fourth stimulus. Vertical dashed lines show time points of interest, as described herein.

At t0, a first dose of NMBA is administered (e.g., to the patient 10 of FIGS. 1-2). When NMBA is first administered, 0% of ACh receptors may be blocked. As ACh receptors may not be immediately blocked (e.g., time may pass as NMBA is absorbed by the patient and begins to block ACh receptors), the patient may respond to TOF stimulation. A TOF measurement is taken that includes four stimuli. Magnitudes of the response to a fourth stimulus (T4) and response to a first stimulus (T1) are equal, thus TOF ratio is equal to one (e.g., T4/T1=1). Between t0 and t1, NMBA begins to block ACh receptors such that a percentage of blocked ACh receptors increases. The percentage of blocked ACh receptors is less than the monitoring margin line 412.

At t1, the percentage of blocked ACh receptors reaches and exceeds the monitoring margin line 412. The TOF ratio begins to decrease; as an increasing percentage of total ACh receptors are blocked, muscle activation in response to TOF stimulus decreases. Between t1 and t2, the percentage of blocked ACh receptors continues to increase, and the TOF ratio continues to decrease. While there is muscle activation in response to the fourth stimulus (T4), TOF ratio may be a sufficient NMT monitoring tool (e.g., TOF ratio may be used to monitor NMT between t1 and t2).

At t2, the TOF ratio is 0 (e.g., equal to the stimulation response line 414), indicating there is no muscle activation to the fourth stimulus of TOF stimulation train (e.g., T4=0, 0/T1=0). A desirable percentage of ACh receptors are blocked (e.g., the percentage of blocked ACh receptors is equal to the stimulation response line 414), and the patient may be sufficiently relaxed. Between t2 and t4, the TOF ratio is zero, and the percentage of blocked ACh receptors may continue to increase (e.g., greater than the stimulation response line 414).

At t3, the percentage of blocked ACh receptors may begin to decrease, and the TOF ratio may still be zero. During this time period between t2 and t4, it may be desirable to monitor NMT using a TOF count and post-tetanic count (PTC). Briefly PTC monitoring differs from TOF monitoring in that PTC monitoring applies a tetanic nerve stimulation to the patient for a first duration, followed by a rest period, and then a series of single twitch stimuli.

At t4, the percentage of blocked ACh receptors percentage decreases below the stimulation response line 414. The patient may again experience muscle activation in response to the fourth stimulus. The TOF ratio begins to increase and is greater than the stimulation response line 414. The percentage of blocked ACh receptors continues to decrease between t4 and t5, and the TOF ratio continues to increase between t4 and t5 (e.g., increasing muscle activation in response to the fourth stimulus). Between t4 and t5, TOF stimulation may be a sufficient tool for monitoring NMT response.

At t5, the percentage of blocked ACh receptors reaches and continues to decrease to less than the monitoring margin line 412. For example, 70% or less of ACh receptors may be blocked by NMBA. The TOF ratio is equal to 1, indicating 100% equal responses to the first stimulus and the fourth stimulus. However, when the percentage of ACh receptors that are blocked is at and/or below the monitoring margin line 412, there may still be significant residual neuromuscular block. Thus, a maximum ability of TOF monitoring (e.g., TOF ratio is 1) may be reached prior to full recovery of the patient from skeletal muscle paralysis (e.g., neuromuscular receptors may still be blocked). Thus, it is desirable to monitor NMT using a method other than TOF monitoring. As is further described herein, tetanic fade may be used for NMT monitoring at and beyond t5.

FIG. 5 is a flow chart of a method 500 for selective application of tetanic stimulus to monitor NMT during patient recovery following administration of NMBA. The method 500 is a detailed example of the method 300, and includes similar operations. Tetanic stimulus is supplied to a nerve of the patient for a preset duration, and evoked response of a muscle innervated by the nerve is continuously monitored. If fade in the response is detected (e.g., decrease in muscle activity), the tetanic stimulus is automatically aborted preemptively before the preset duration of tetanic stimulus is expired. Different NMT modalities can be used for monitoring the response, including EMG, KMG, MMG, AMG, PMG, and so on. The method 500 may be carried out according to instructions stored on a computing system, including but not limited to the processor 128 of the patient care and monitoring system 100 of FIGS. 1-2. Method 500 may be executed when sensors of the patient care and monitoring system 100 are securely connected to their respective electrodes on the patient's forearm, such as shown in FIG. 2.

At 502, the method 500 includes starting application of a tetanic stimulus and applying the tetanic stimulus for a first preset duration via a peripheral nerve stimulator. The first preset duration may be five seconds, for example. In other examples, the first preset duration may be more than or less than five seconds. The first preset duration starts at time (t)=0.

At 504, the method 500 includes measuring NMT response at t>0 (e.g., registering an initial physiological response at a start of tetanic stimulus) via a physiological response monitor. For example, the NMT response may be measured as muscle activity is monitored at the start of the first duration and throughout the duration. There may be a delay (e.g., approximately 3 ms from nerve stimulation at t=0 ms) to an onset of muscle activation. A duration of the delay may be characteristic of a NMT monitoring modality (e.g., EMG, KMG, MMG, AMG, PMG, and so on). Thus, a time t>0 at which the initial NMT response is measured may be configurable based on monitoring modality.

At 506, the method 500 includes measuring NMT response at time t=n * Δt, for n=1, 2, 3, . . . N via the physiological response monitor. In this way, NMT response is continuously measured. A frequency at which the NMT response is measured may be a fraction of the preset duration for which the tetanic stimulus is applied.

At 508, the method 500 includes determining if the NMT response at tₙ is greater than the NMT response at t>0 (e.g., the initial NMT response). As NMT response is measured, the NMT response is compared to the NMT response at t>0 in real-time, enabling the tetanic stimulus to be automatically preemptively aborted quickly when it is determined the NMT response is different than the initial NMT response.

In response to the NMT response at tₙ being smaller than the NMT response at t>0, (YES at 508) the method 500 proceeds to 510. At 510, the method 500 includes automatically preemptively aborting tetanic stimulus administered by the peripheral nerve stimulator at time tₙ.

The method 500 proceeds to 512 and halts further application of tetanic stimulus for a second preset duration. The second preset duration may be a rest period in which tetanic stimulus is not applied, which may enable the patient to recover from prior tetanic stimulus, thus reducing stress and/or pain experienced by the patient. Further, NMBA may evacuate the NMT receptor in the second preset duration, such that the percentage of ACh receptors that are not blocked increases. The second preset duration may be significantly longer than the first preset duration, such as five minutes. The method 500 returns to operation 502 to continue to apply a next tetanic stimulus.

Returning to operation 508, in response to the NMT response at tₙ not being smaller than t>0, (NO at 508), the method 500 proceeds to 514 to continue administering tetanic stimulus via the peripheral nerve stimulator for the remaining time of the first preset duration.

At 516, the method 500 includes determining if tₙ is less than the first preset duration. Described another way, the method 500 includes determining if the patient has not exhibited an NMT response for the entirety of the first preset duration, or if there is remaining time in the preset duration. In response to tₙ being less than the preset duration (YES at 516), the method 500 returns to operation 506 to continue measuring the NMT response while tetanic stimulus is administered.

In response to tₙ not being less than the first preset duration (e.g., greater than or equal to the first preset duration, NO at 516), the method 500 proceeds to 518. At 518, the method 500 includes preemptively aborting tetanic stimulus provided by the peripheral nerve stimulator, as the first preset duration for tetanic stimulus administration is ended. The method 500 ends.

FIG. 6 shows a timeline graph 600 of tetanic stimulus 602 and muscle activity 604 in response to tetanic stimulus. The graph 600 may be an example of execution of the method 500. For example, tetanic stimulus may be applied to a patient via a NMT monitoring device, such as the patient care and monitoring system 100 of FIGS. 1-2, in accordance with the operations of the method 500. Muscle activity may be monitored using the same device that is used to administer the tetanic stimulus. The method 500 may be implemented to monitor recovery of a patient following a surgical procedure and, specifically, to monitor decrease of NMT blockage by NMBA. Thus, the timeline graph 600 may show tetanic stimulus and resulting muscle activity when NMBA is not being administered to the patient. The plot of tetanic stimulus 602 shows whether tetanic stimulus is on or off along the vertical axis. The plot of muscle activity 604 shows relative muscle activity, from high to low, along the vertical axis. Time increases from left to right along the horizontal axis. In the method 500 of FIG. 5, tetanic stimuli are applied at constant intervals t0, t 1. . . tn, such as 0 ms, 10 ms, and so on. In the timeline graph 600 of FIG. 6, events of interest are indicated at times t0, t1. . . t5. It is to be understood that time indicators of FIGS. 5 and 6 may be different from each other and may not indicate a same moment in time.

Between t0 and t1, tetanic stimulus is not applied to the patient; tetanic stimulus 602 is off. Muscle activity 604 is low. Because nerves are not being stimulated by the tetanic stimulus and may still be experiencing effects of NMBA administered prior to and/or during the surgical procedure (e.g., ACh receptors may be blocked), muscle activity may be low.

At t1, tetanic stimulus is applied to the patient (e.g., tetanic stimulus is on), and continuously measured muscle activity in response to tetanic stimulus is high. Muscle activity at t1 is registered, and muscle activity after t1 is monitored and compared to muscle activity at t1 to detect tetanic fade that may indicate residual NMT blockage. The method 500 may be configured to apply the tetanic stimulus for a first preset duration, such as five seconds, or until a detected change in physiological response automatically and prematurely aborts tetanic stimulus.

At t2, muscle activity begins to decrease. Muscle activity decrease may indicate that NMT, such as ACh receptors, may still be blocked by NMBA. Muscle activity decrease may occur before the first preset duration ends. Where t1 is a start of tetanic stimulus application (e.g., time (t)=0), t2 may be less than five seconds (e.g., t2=2 seconds). If ACh receptors are sufficiently free from NMBA blockage, muscle activity may be maintained by the muscle for the entire duration of the preset duration in which tetanic stimulus is applied. If a significant number of ACh receptors are blocked (e.g., as described with respect to FIG. 3), muscle activity may start to fade, such as within one second after the onset of tetanic stimulus.

Detection of a decrease in muscle activity, relative to muscle activity at t1, triggers preemptive abortion of the tetanic stimulus (e.g., prior to the end of the first preset duration). Muscle activity is continuously measured and analyzed, and detection takes place if a prolonged decrease in a muscle activity trend is observed. At t3, detection is confirmed and application of the tetanic stimulation is aborted. A duration between detection of the decrease in muscle activity and abortion of application of the tetanic stimulus (e.g., between t2 and t3) may be configurable. It may be desirable for the duration to be as short as possible. For example, the duration may be one second. In other examples, the duration may be one millisecond.

Following preemptive abortion of the tetanic stimulus, tetanic stimulus may not be applied for a second preset duration (e.g., between t3 and t4). The second preset duration may be a duration in which the patient may recover from pain and/or stress caused by tetanic stimulus. Additionally, more ACh receptors may become unblocked during the second preset duration. The second preset duration may be a configurable length. For example, the second preset duration may be longer or than the preset duration (e.g., between two and twenty minutes).

At t4, tetanic stimulus is again applied to the subject to assist in monitoring NMT blockage. The tetanic stimulus may be applied at the same intensity as is applied at t1. In response to tetanic stimulus 602 at t4, muscle activity 604 increases. The tetanic stimulus may further be applied for the first preset duration, and may be preemptively aborted in response to detection of a decrease in muscle activity. For example, the duration between t4 and t5 may be the first preset duration. Between t4 and t5, and thus for the entirety of the first preset duration, muscle activity may not decrease. This may indicate that a sufficient percentage of ACh receptors are not blocked, and the patient is sufficiently recovered from NMBA. At the end of the first preset duration (e.g., at t5), tetanic stimulus is preemptively aborted, and muscle activity in response to tetanic stimulus is low.

FIG. 7 is a flow chart of a method 700 for selective application of tetanic stimulus to monitor nociceptive response (e.g., vasoconstriction) during administration of analgesic medication to patients, such as prior to and/or during a surgical procedure. The method 700 is a detailed example of the method 300, and includes similar operations. Tetanic stimulus is supplied to a nerve of the patient for a preset duration, and an evoked nociceptive response, such as vasoconstriction, is continuously monitored. If a nociceptive response is detected (e.g., increased vasoconstriction), the tetanic stimulus is automatically aborted preemptively (e.g., before the preset duration of tetanic stimulus is expired). Vasoconstriction may be monitored by one or more physiological response monitors (e.g., the physiological response monitor 130 of FIGS. 1-2). For example, the physiological response monitor may be configured as a SpO₂ monitor, PPG pulse wave monitor, or other device configured to monitor vasoconstriction. The method 700 may be carried out according to instructions stored on a computing system, including but not limited to the processor 128 of the patient care and monitoring system of FIGS. 1-2. Method 700 may be executed when sensors of the patient care and monitoring system 100 are securely connected to their respective electrodes on the patient's forearm, such as shown in FIG. 2.

At 702, the method 700 includes starting application of a tetanic stimulus and applying the tetanic stimulus for a first preset duration via a peripheral nerve stimulator. The first preset duration may be five seconds, for example. In other examples, the first preset duration may be more than or less than five seconds. The first preset duration starts at time (t)=0.

At 704, the method 700 includes measuring vasoconstriction at t=0 (e.g., registering an initial physiological response at a start of tetanic stimulus) via a physiological response monitor. For example, the NMT response may be measured as vasoconstriction is monitored at the start of the first duration and throughout the duration.

At 706, the method 700 includes measuring vasoconstriction at time t=n * Δt, for n=1, 2, 3, . . . N via the physiological response monitor. In this way, vasoconstriction is continuously measured. As described with respect to FIG. 4, a frequency at which the vasoconstriction is measured may be a fraction of the preset duration for which the tetanic stimulus is applied.

At 708, the method 700 includes determining if the vasoconstriction at tₙ is greater than the vasoconstriction at t₀. As vasoconstriction is measured, the vasoconstriction is compared to the vasoconstriction at t₀ in real-time, enabling the tetanic stimulus to be automatically preemptively aborted quickly when it is determined the vasoconstriction is different than the initial vasoconstriction.

In response to the vasoconstriction at tₙ being greater than the vasoconstriction at t₀, (YES at 708) the method 700 proceeds to 710. At 710, the method 700 includes automatically preemptively aborting tetanic stimulus applied by the peripheral nerve stimulator at time tₙ.

The method 700 optionally includes administering an analgesic drug at 712. The increased vasoconstriction may be caused by insufficient sedation of the patient. The method 700 may prompt administration of an additional dose of analgesic drug and/or the dose of analgesic may be increased. In another example, the method 700 may automatically determine a desired analgesic dose and may administer the desired analgesic dose to the patient.

The method 700 proceeds to 714 and halts further application of tetanic stimulus for a second preset duration. The second preset duration may be a rest period in which tetanic stimulus is not applied, which may enable the patient to recover from prior tetanic stimulus, thus reducing stress and/or pain experienced by the patient. Further, the analgesic dose may take effect during the second preset duration. The method 700 returns to operation 706 to continue to measure NMT responses.

Returning to operation 708, in response to the vasoconstriction at tₙ not being greater than t₀, (NO at 708), the method 700 proceeds to 716 to continue administering tetanic stimulus for the remaining time of the first preset duration.

At 718, the method 700 includes determining if tₙ is less than the first preset duration. Described another way, the method 700 includes determining if the patient has not exhibited an increase in vasoconstriction for the entirety of the first preset duration, or if there is remaining time in the preset duration. In response to tₙ being less than the preset duration (YES at 718), the method 700 returns to operation 706 to continue measuring vasoconstriction while tetanic stimulus is administered.

In response to tₙ not being less than the first preset duration (e.g., greater than or equal to the first preset duration, NO at 718), the method 700 proceeds to 720. At 720, the method 700 includes preemptively aborting tetanic stimulus administration via the peripheral nerve stimulator, as the first preset duration for tetanic stimulus administration is ended. The method 700 includes, at 722, outputting for display and/or as an output signal, an indication that sufficient dose of analgesic has been delivered to the patient to sufficiently sedate the patient. For example, an indicator of sufficient analgesic may be output to a display unit and/or a memory. The method 700 ends.

FIGS. 8 and 9 shows a first graph 800 and a second graph 900, respectively, each illustrating a photoplethysmographic (PPG) response to two successive tetanic stimuli over time (in seconds). FIGS. 8 and 9 are described jointly herein, with reference to each graph noted. Each tetanic stimulus may be 5 seconds(s) long, and tetanic stimuli may be applied at a 100 s interval. The first graph 800 shows a first tetanic stimulus, and the second graph 900 shows a second tetanic stimulus, where the second tetanic stimulus is administered after (e.g., 100 s after) the first tetanic stimulus. If present, vasoconstriction is an immediate response after the onset of tetanic stimulus. Vasoconstriction is especially visible in a DC component of a PPG response.

A first tetanic stimulus is applied at approximately a first time point 802. The PPG signal increases (e.g., has a rising slope) for a first duration 804. The PPG signal increases for approximately the full duration of the five second stimulus (e.g., a length of the first duration 804). The PPG signal decreases for a second duration 806 after the tetanic stimulus ends. The second duration 806 may be equal to 100 s.

Turning to FIG. 9, a second tetanic stimulus is applied at approximately a second time point 902. The PPG signal increases (e.g., has a rising slope) for a third duration 904. The PPG signal increases for approximately the full duration of the five second stimulus (e.g., a length of the third duration 904). The PPG signal decreases for a fourth duration 906 after the tetanic stimulus ends. The fourth duration 906 may be equal to 100 s. As the PPG signal continues to increase for almost the full duration of the tetanic stimulus, it is justified to expect that preemptively aborting the stimulus prior to 5 seconds may cause milder vasoconstriction being less stressful to the patient.

FIG. 10 shows a timeline graph 1000 of tetanic stimulus 1002 and nociceptive response 1004 in response to tetanic stimulus. The graph 1000 may be an example of execution of the method 700. For example, tetanic stimulus may be applied to a patient via a nociceptive response monitoring device, such as the patient care and monitoring device 100 of FIGS. 1-2, in accordance with the operations of the method 700. Nociceptive response may be monitored using the same device that is used to administer the tetanic stimulus. The method 700 may be implemented to monitor analgesic administration to a patient prior to and/or during a surgical procedure. Thus, the timeline graph 1000 may show tetanic stimulus and resulting nociceptive response after administering analgesic to the patient. The plot of tetanic stimulus 1002 shows whether tetanic stimulus is on or off along the vertical axis. The plot of nociceptive response 1004 shows relative nociceptive response, from high to low, along the vertical axis. The nociceptive response may be, for example, a vasoconstriction response, EMG, or another fast sympathetic reflex response. In some examples, a quantified nociceptive response (e.g., instead of binary yes/no) can be utilized to control the tetanic abortion and/or proposed analgesic dose. For example, a timing of tetanic abortion and/or a dosage of the analgesic drug may be determined in response to a magnitude of the nociceptive response. In the method 700 of FIG. 7, tetanic stimuli are applied at constant intervals t0, t1. . . tn. In the timeline graph 1000 of FIG. 10, events of interest are indicated at times t0, t1. . . t5. It is to be understood that time indicators of FIGS. 7 and 10 may be different from each other and may not indicate a same moment in time.

Between t0 and t1, tetanic stimulus is not applied to the patient; tetanic stimulus 1002 is off. Nociceptive response 1004 is low. Because nerves are not being stimulated by the tetanic stimulus, there may be no/low nociceptive response.

At t1, tetanic stimulus is applied to the patient (e.g., tetanic stimulus is on). Nociceptive response to the tetanic stimulus is low. For example, a first analgesic dose may be applied to the patient between t0 and t1, and a nociceptive response of the patient may be at least partially blocked (e.g., antinociception induced by analgetic drugs) at t1. As described with respect to FIG. 7, nociceptive response at t1 is registered, and nociceptive response after t1 is monitored and compared to nociceptive response at t1 to detect an increase in nociceptive response that may indicate the patient is not sufficiently sedated. The method 700 may be configured to apply the tetanic stimulus for a first preset duration, such as five seconds, or until a detected change in physiological response automatically and prematurely aborts tetanic stimulus.

At t2, tetanic stimulus is still applied to the patient (e.g., at the same level it was applied at t1). Nociceptive response of the patient increases. A desirable level of nociceptive response block may not be achieved, therefore the patient may sense and respond to the tetanic stimulus.

In response to nociceptive response increasing, at t3, tetanic stimulus is automatically preemptively turned off. Between t3 and t4, nociceptive response gradually decreases. In some examples, an additional dose of analgesic may be applied and/or the dose of analgesic may be increased between t4 and t5. In further examples, analgesic dose may not be increased, and instead administration of tetanic stimulus may be delayed for a second preset duration to allow analgesic to work to block receptors, as well as allow the subject to recover from tetanic stimulus.

At t5, the tetanic stimulus is applied for the first preset duration. Nociceptive response is low and remains low for the first duration (e.g., between t5 and t6). This may indicate that the subject is sufficiently sedated and sufficient analgesic has been applied. At t6, tetanic stimulus is turned off, and the method ends. Surgery or other medical procedure may proceed.

In this way, automatic and immediate abort of on-going technical stimulus may be achieved based on a monitored physical response of the subject. Real-time monitoring of evoked nociceptive and/or muscle response and administration of tetanic stimulus by a single system reduces complexity and response (e.g., tetanic stimulus abortion) delays.

Technical benefits include enabled NMT monitoring in clinically desirable ranges, such as when 0-70% of ACh receptors are still blocked that conventional monitoring devices cannot effectively measure. Further, the methods and systems provided herein provide integration of multiple measurement modalities in a single system and method, thus providing expanded measurement capabilities through real-time monitoring. The systems and methods described herein provide more precise control than manual methods.

The disclosure also provides support for a method, comprising: applying a tetanic stimulus to a nerve of a patient for a preset duration via a peripheral nerve stimulator of a patient care and monitoring system, measuring and registering an initial physiological response of the patient at a start of the tetanic stimulus via a physiological response monitor of the patient care and monitoring system, monitoring a physiological response of the patient periodically within the preset duration via the physiological response monitor, and automatically preemptively aborting the tetanic stimulus administered by the peripheral nerve stimulator based on a comparison of the physiological response to the initial physiological response. In a first example of the method, the tetanic stimulus is preemptively aborted in response to the physiological response decreasing within the preset duration with respect to the initial physiological response. In a second example of the method, optionally including the first example, the physiological response is muscle activity, the physiological response monitor is configured to measure muscle activity, and tetanic stimulus is applied via the peripheral nerve stimulator following administration of neuromuscular blocking agents to the patient. In a third example of the method, optionally including one or both of the first and second examples, the tetanic stimulus is preemptively aborted in response to the physiological response increasing within the preset duration with respect to the initial physiological response. In a fourth example of the method, optionally including one or more or each of the first through third examples, the physiological response is nociceptive response, the physiological response monitor is configured to measure nociceptive response, and tetanic stimulus is applied following administration of analgesic drugs. In a fifth example of the method, optionally including one or more or each of the first through fourth examples, the method further comprises: adjusting a dose of analgesic drugs via an administration system of the patient care and monitoring system after the tetanic stimulus is automatically preemptively aborted. In a sixth example of the method, optionally including one or more or each of the first through fifth examples, the preset duration is five minutes. In a seventh example of the method, optionally including one or more or each of the first through sixth examples, the method further comprises: following preemptive abortion of the tetanic stimulus, halting administration of the tetanic stimulus for a second preset duration, and applying the tetanic stimulus via the peripheral nerve stimulator after the second preset duration. In an eighth example of the method, optionally including one or more or each of the first through seventh examples, the second preset duration is five seconds. In a ninth example of the method, optionally including one or more or each of the first through eighth examples, the physiological response is based on an electromyography signal, mechanomyography signal, phonomyography signal, kinemyography signal, acceleromyography signal, photoplethysmography signal, and/or vasoconstriction.

The disclosure also provides support for a method for patient monitoring before, during, and/or after a surgical procedure, comprising: applying a tetanic stimulus to a nerve of a patient for a first preset duration via a peripheral nerve stimulator of a patient care and monitoring system, measuring and registering an initial physiological response of the patient at a start of the tetanic stimulus via a physiological response monitor of the patient care and monitoring system, monitoring a physiological response of the patient periodically within the first preset duration via the physiological response monitor, automatically preemptively aborting the tetanic stimulus administered by the peripheral nerve stimulator based on a comparison of the physiological response to the initial physiological response indicating that the physiological response is different from the initial physiological response, halting application of the tetanic stimulus by the peripheral nerve stimulator for a second preset duration, and applying the tetanic stimulus to the nerve of the patient via the peripheral nerve stimulator for the first preset duration via the peripheral nerve stimulator until the physiological response is not different from the initial physiological response. In a first example of the method, monitoring the physiological response of the patient periodically within the first preset duration includes measuring the physiological response at a frequency that is a fraction of the first preset duration. In a second example of the method, optionally including the first example, the second preset duration is longer than the first preset duration. In a third example of the method, optionally including one or both of the first and second examples, the method further comprises: determining a desired dose of an analgesic drug that sufficiently causes the physiological response to be the same as the initial physiological response during the first preset duration, and administering the analgesic drug to the patient via an administration system of the patient care and monitoring system. In a fourth example of the method, optionally including one or more or each of the first through third examples, the method further comprises: outputting an indication of sufficient analgesia in response to the physiological response being the same as the initial physiological response when the physiological response is used to monitor administration of analgesic drugs.

The disclosure also provides support for a patient care and monitoring system, comprising: a peripheral nerve stimulator, a physiological response monitor, and a host patient monitor comprising a processor and a memory storing instructions that, when executed by the processor, cause the host patient monitor to: apply a tetanic stimulus to a nerve of a patient for a preset duration via the peripheral nerve stimulator, register an initial physiological response of the patient, detected by the physiological response monitor, at a start of the tetanic stimulus, monitor a physiological response of the patient periodically within the preset duration via the physiological response monitor, and automatically preemptively abort the tetanic stimulus based on a comparison of the physiological response to the initial physiological response. In a first example of the system, the physiological response monitor is an electromyography sensor that comprises a plurality of sensing electrodes. In a second example of the system, optionally including the first example, the host patient monitor is configured to receive user input indicating a duration of the preset duration, and applying the tetanic stimulus for the preset duration. In a third example of the system, optionally including one or both of the first and second examples, the physiological response monitor includes at least one of an electromyography (EMG) sensor, mechanomyography (MMG) sensor, phonomyography (PMG) sensor, kinemyography (KMG) sensor, acceleromyography (AMG) sensor, and/or photoplethysmography (PPG) sensor. In a fourth example of the system, optionally including one or more or each of the first through third examples, the system further comprises: an administration system configured to administer analgesic drugs to the patient in response to indication that the patient is in insufficient analgesia, based on comparison of the physiological response with the initial physiological response.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
applying a tetanic stimulus to a nerve of a patient for a preset duration via a peripheral nerve stimulator of a patient care and monitoring system;
measuring and registering an initial physiological response of the patient at a start of the tetanic stimulus via a physiological response monitor of the patient care and monitoring system;
monitoring a physiological response of the patient periodically within the preset duration via the physiological response monitor; and
automatically preemptively aborting the tetanic stimulus administered by the peripheral nerve stimulator based on a comparison of the physiological response to the initial physiological response.

2. The method of claim 1, wherein the tetanic stimulus is preemptively aborted in response to the physiological response decreasing within the preset duration with respect to the initial physiological response.

3. The method of claim 2, wherein the physiological response is muscle activity, the physiological response monitor is configured to measure muscle activity, and tetanic stimulus is applied via the peripheral nerve stimulator following administration of neuromuscular blocking agents to the patient.

4. The method of claim 1, wherein the tetanic stimulus is preemptively aborted in response to the physiological response increasing within the preset duration with respect to the initial physiological response.

5. The method of claim 4, wherein the physiological response is nociceptive response, the physiological response monitor is configured to measure nociceptive response, and tetanic stimulus is applied following administration of analgesic drugs.

6. The method of claim 5, further comprising adjusting a dose of analgesic drugs via an administration system of the patient care and monitoring system after the tetanic stimulus is automatically preemptively aborted.

7. The method of claim 1, wherein the preset duration is five minutes.

8. The method of claim 1, further comprising, following preemptive abortion of the tetanic stimulus, halting administration of the tetanic stimulus for a second preset duration, and applying the tetanic stimulus via the peripheral nerve stimulator after the second preset duration.

9. The method of claim 8, wherein the second preset duration is five seconds.

10. The method of claim 1, wherein the physiological response is based on an electromyography signal, mechanomyography signal, phonomyography signal, kinemyography signal, acceleromyography signal, photoplethysmography signal, and/or vasoconstriction.

11. A method for patient monitoring before, during, and/or after a surgical procedure, comprising:
applying a tetanic stimulus to a nerve of a patient for a first preset duration via a peripheral nerve stimulator of a patient care and monitoring system;
measuring and registering an initial physiological response of the patient at a start of the tetanic stimulus via a physiological response monitor of the patient care and monitoring system;
monitoring a physiological response of the patient periodically within the first preset duration via the physiological response monitor;
automatically preemptively aborting the tetanic stimulus administered by the peripheral nerve stimulator based on a comparison of the physiological response to the initial physiological response indicating that the physiological response is different from the initial physiological response;
halting application of the tetanic stimulus by the peripheral nerve stimulator for a second preset duration; and
applying the tetanic stimulus to the nerve of the patient via the peripheral nerve stimulator for the first preset duration via the peripheral nerve stimulator until the physiological response is not different from the initial physiological response.

12. The method of claim 11, wherein monitoring the physiological response of the patient periodically within the first preset duration includes measuring the physiological response at a frequency that is a fraction of the first preset duration.

13. The method of claim 11, wherein the second preset duration is longer than the first preset duration.

14. The method of claim 11, further comprising determining a desired dose of an analgesic drug that sufficiently causes the physiological response to be the same as the initial physiological response during the first preset duration, and administering the analgesic drug to the patient via an administration system of the patient care and monitoring system.

15. The method of claim 11, further comprising outputting an indication of sufficient analgesia in response to the physiological response being the same as the initial physiological response when the physiological response is used to monitor administration of analgesic drugs.

16. A patient care and monitoring system, comprising:
a peripheral nerve stimulator;
a physiological response monitor; and
a host patient monitor comprising a processor and a memory storing instructions that, when executed by the processor, cause the host patient monitor to:
apply a tetanic stimulus to a nerve of a patient for a preset duration via the peripheral nerve stimulator;
register an initial physiological response of the patient, detected by the physiological response monitor, at a start of the tetanic stimulus;
monitor a physiological response of the patient periodically within the preset duration via the physiological response monitor; and
automatically preemptively abort the tetanic stimulus based on a comparison of the physiological response to the initial physiological response.

17. The patient care and monitoring system of claim 16, wherein the physiological response monitor is an electromyography sensor that comprises a plurality of sensing electrodes.

18. The patient care and monitoring system of claim 16, wherein the host patient monitor is configured to receive user input indicating a duration of the preset duration, and applying the tetanic stimulus for the preset duration.

19. The patient care and monitoring system of claim 16, wherein the physiological response monitor includes at least one of an electromyography (EMG) sensor, mechanomyography (MMG) sensor, phonomyography (PMG) sensor, kinemyography (KMG) sensor, acceleromyography (AMG) sensor, and/or photoplethysmography (PPG) sensor.

20. The patient care and monitoring system of claim 16, further comprising an administration system configured to administer analgesic drugs to the patient in response to indication that the patient is in insufficient analgesia, based on comparison of the physiological response with the initial physiological response.
